(19) United States
(12) Patent Application Publication
(10) Pub. No.: US 2009/0149756 A1
(43) Pub. Date: Jun. 11, 2009

(54) METHOD, APPARATUS AND COMPUTER PROGRAM FOR THREE-DIMENSIONAL ULTRASOUND IMAGING

(75) Inventors: Pau Soler, Barcelona (ES); Olivier Gerard, Viroflay (FR); Gaspar Delso, Puteaux (FR); Pascal Allain, Saint Cyr L'ecole (FR)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS, N.V., EINDHOVEN (NL)

(21) Appl. No.: 12/304,286

(22) PCT Filed: Jun. 19, 2007
(86) PCT No.: PCT/IB2007/052336

§ 371 Date: Dec. 11, 2008

(30) Foreign Application Priority Data
Jun. 23, 2006 (EP) 06300720.7

(51) Int. Cl. A61B 8/14

(52) U.S. Cl. 600/443

(57) ABSTRACT

A method and apparatus for medical ultrasound imaging. A three dimensional image data of a whole organ is acquired at a first resolution during a cardiac cycle. A three dimensional image data of a sector of the organ is acquired at a second higher resolution during another cardiac cycle. The data are compared so as to allow registration of the sector with respect to the whole organ.

### FIELD OF THE INVENTION

This invention relates to medical ultrasound imaging and, more particularly, to a method, an apparatus and a computer program for three-dimensional ultrasound imaging.

### BACKGROUND OF THE INVENTION

Three-dimensional echocardiology has many benefits in medical diagnosis in comparison to classical two-dimensional imaging. For instance, it allows more accurate quantification such as left ventricle volume measurement and ejection fraction computation, since no hypotheses are made on the shape of the left ventricle. Moreover, the overall examination time is reduced as most parts of the heart are visible within a single acquisition.

On the other hand, since the amount of data to be collected is larger than in the two-dimensional case, both spatial and temporal resolution are reduced. To obtain high-resolution large volumes, the acquisition of the left ventricle is usually divided in four sectors, each sector being obtained in a different cardiac cycle. However, this need of several cardiac cycles to obtain high-resolution volume may cause disjunctions between different sectors of the data if movement of heart is not exactly periodic during the several cardiac cycles.

As an example, medical ultrasound three-dimensional imaging system according to U.S. Pat. No. 5,993,390 acquires ultrasound image data representative of three-dimensional sectors of a volume of interest in a patient in synchronism with corresponding cardiac cycles of the patient, and combines the image data representative of the sectors to provide image data representative of a three-dimensional ultrasound image of the volume.

As mentioned in this patent, a disadvantage of the system is that motion of the heart between heartbeats may cause discontinuities in the displayed image.

### SUMMARY OF THE INVENTION

An aim of this invention is to provide an acquisition method that allows avoiding junction artefacts in a three-dimensional image of a volume of interest.

According to the invention, a method for medical ultrasound imaging, comprises the steps of:

a) acquiring with a first resolution a first ultrasound image data representative of a volume of an interest organ during a cardiac cycle of a patient;
b) acquiring with a second resolution higher than the first resolution a second ultrasound image data representative of a three-dimensional sector of said volume during another cardiac cycle of the patient;
c) comparing the first and the second ultrasound image data of said sector so as to detect a similarity between the first and the second ultrasound image data and, as a function of said comparison,
validating the three-dimensional ultrasound second image data if the first and the second ultrasound image data are substantially similar or otherwise
further processing said sector.

Acquiring with a first, low resolution allows obtaining in a single cardiac cycle an image of the whole volume of interest. This image is used as a reference for the sectors acquired at the second, higher resolution. By comparing a sector acquired at high resolution with the corresponding portion of the volume acquired at low resolution, it can be assessed if the sector has moved or not between the acquisition of the volume and the acquisition of the sector. This information allows avoiding artifacts in the final high-resolution image, because a sector that has moved between the acquisition of the volume and the acquisition of the sector creates artifact in the final high-resolution image if it is not further processed.

The invention also relates to an apparatus for medical ultrasound imaging, as well as to a computer program for implementing the method in accordance with the invention.

### BRIEF DESCRIPTION OF THE DRAWINGS

The present invention will now be described in more detail, by way of example, with reference to the accompanying drawings, wherein:

FIG. 1 is a cross-sectional view of a three-dimensional volume (FIG. 1A) that is divided into sectors (FIG. 1B);

FIG. 2 is a schematic representation of a three-dimensional volume displaying artefacts;

FIG. 3 shows an ECG waveform that is divided into twenty cardiac phases;

FIG. 4 shows a flow diagram of acquisition of the images according to the invention.

### DETAILED DESCRIPTION

All of the known prior art three-dimensional cardiac imaging techniques have had low resolution and/or long acquisition times. In the case of long acquisition times, the images typically exhibit discontinuities due to cardiac, respiratory, patient and/or sonographer movement. As mentioned before, due to the need of high resolution, echocardiography full-volume acquisitions are currently performed within a plurality of cardiac cycles, for instance four. At each cycle, a sector of only one fraction (for instance one fourth) of the whole cardiac volume is imaged. The imaged cardio-vascular organ may also be only the left ventricle, but still it is imaged using four different sectors. It should be noticed that other interest organs may be imaged in accordance with the invention, as soon as their movement has a certain periodicity which is a function of the cardiac cycle.

However, any movement of the patient (breath, displacement), of the ultrasound probe or any heart rate alteration (i.e. arrhythmia) results in a boundary between sectors and generate artefacts (F) as shown by arrow on FIG. 2.

It should be noted that US 2005/0228280 discloses a method for acquiring ultrasound data for display. The method comprises:

(a) scanning within a three-dimensional volume with a first spatial resolution; and
(b) scanning within a three-dimensional sub-volume of the volume with a second spatial resolution, the second spatial resolution being higher than the first spatial resolution.

In more details, step (b) comprises scanning within the entire three-dimensional sub-volume at the second spatial resolution and step (a) comprises scanning at the first spatial resolution within the entire three-dimensional volume other than the sub-volume.

A lower spatial resolution volume may be more rapidly scanned than a same region with a higher spatial resolution. By scanning the sub-volume with higher spatial resolution, medical diagnosis may be improved or based on more information content. However such medical diagnosis implies that the user have a sufficient medical knowledge to use the image. By scanning the remainder of the three-dimensional volume with a lower resolution, anatomical reference information at a lower resolution may be provided for helping the operator to position the sub-volume associated with higher resolution imaging. However, this document does not disclose that the image of a sub-volume acquired at high resolution is compared to the image of the same sub-volume acquired at lower resolution. In addition, the three-dimensional sub-volume and the three-dimensional volume other than said sub-volume refer to two distinct regions of the volume to be imaged and thus artefacts may occur at boundaries between said volume and sub-volume.

The invention described hereafter is an acquisition method to obtain large high-resolution image volumes without artefacts, the apparatus for implementing such method and the computer program to control the operation of the apparatus.

The apparatus for medical ultrasound imaging comprises:
a transducer comprising an array of transducer elements;
a transmitter for transmitting ultrasound energy with said transducer into a volume of an interest organ as a plurality of transmit beams;
a receiver for receiving ultrasound echoes with said transducer from the volume in response to said ultrasound energy and for generating received signals representative of the received ultrasound echoes;
a receive beamformer for processing said received signals to form at least one receive beam for each of said transmit beams and to generate an image data representative of the ultrasound echoes in said receive beam;
a circuit arranged for controlling acquisition of image data representative of a volume during a cardiac cycle with a first resolution and acquisition of image data representative of a sector of the volume during a cardiac cycle with a second higher resolution, and
a comparator for comparing the first and the second resolution image data of the volume sector.

As a function of said comparison, the apparatus is arranged to validate the three-dimensional ultrasound second resolution image data representative of the sector or to further process the sector.

Additionally, other components may be provided, such as a memory for storage of ultrasound data, a display and a user input. The beamformer is operable to scan within a three-dimensional volume with one spatial resolution and operable to scan within a sub-volume or sector of the three-dimensional volume with a higher spatial resolution. For example, one or more of the frequency of scanning, imaging bandwidth, aperture size, aperture location, apodization type, scan geometry and scan line density are varied depending on which portion of a three-dimensional volume is being scanned. With higher frequency, a larger aperture and a denser scan line distribution, a sub-volume or sector is scanned with a higher spatial resolution. A lower spatial resolution volume may be more rapidly scanned than a same region with a higher spatial resolution.

The beamformer is operable to switch between parameters, such as aperture, frequency, apodization profile, delay profile and combinations thereof between transmit and receive events in order to vary a lateral extent, a scanning position, or resolution. By switching between beamforming parameters any of various combinations of scan patterns may be provided, such as scanning the entire three-dimensional volume in a first resolution mode, for example low resolution, during a first cardiac cycle and then scanning a sub-volume or sector with a second resolution mode, for instance higher resolution, during another cardiac cycle.

The apparatus may use a computer program which control the switching of the beamformer from one resolution mode to a second higher resolution mode, which synchronise the acquisition of the image data with the cardiac cycles signals delivered by, for example an ECG (stands for Electro Cardio Gram) sensor, and which performs the steps of the method in accordance with the invention.

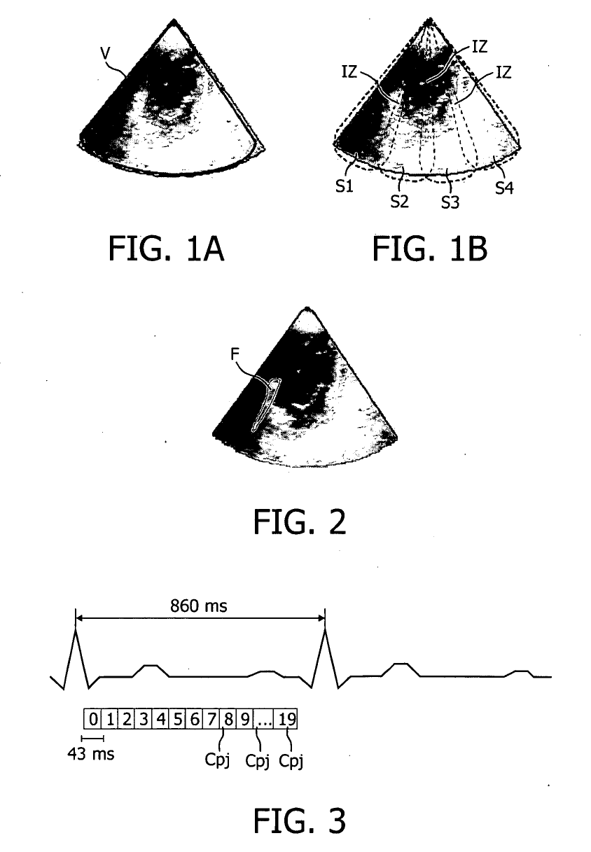

The beamformer signals may be stored in an image data buffer which, as described below, stores image data for different volume sectors (VSi) of an image volume (V) illustrated on FIGS. 1A and 1B (in FIG. 1B the sectors VS₁, VS₂, VS₃ and VS₄ are respectively named S1, S2, S3 and S4), and for different cardiac phases (CPj) of a cardiac cycle Ck illustrated on FIG. 3.

A volume (V) may have a conical shape with an apex centred on the transducer array. A preferred application of the invention is cardiac imaging. To facilitate cardiac imaging, volume (V) may be divided into three-dimensional sectors (VSi) for imaging of the patient's heart.

Thus, for example, the cross-sections of the sectors may be square, rectangular, circular, or irregularly shaped. Furthermore, different sectors may have different sizes and shapes within a single volume.

For a given volume, the selection of the size, shape and number of sectors may be based in part on the time available for image data acquisition during a specified cardiac phase as described below.

It will be understood that the volume itself is not limited to a conical shape and may have a variety of different shapes and sizes. For example, the volume may be a pyramid or a truncated pyramid. The selection of the size and shape of the volume may be based on the application and the type of transducer being utilized

A feature of the invention is based on acquisition of image data for one or more sectors in synchronism with the patient's cardiac cycle. An example of an ECG waveform is shown in FIG. 3. In the example of FIG. 3, ECG waveform indicates a heartbeat every 860 milliseconds. The cardiac cycle may be divided into cardiac phases for imaging. In one example, 20 cardiac phases CPj of approximately 43 milliseconds each may be utilized. The selection of the cardiac phase duration is typically based on the maximum time in which the heart does not move significantly. More or fewer cardiac phases may be utilized.

The ECG waveform of the patient is used to trigger image data acquisition, so that data acquisition is synchronized to each patient's cardiac cycle. More specifically, image data acquisition is synchronized to a specific phase of the cardiac cycle. Furthermore, image data may be acquired during each phase of each cardiac cycle. The amount of image data acquired during each cardiac phase is a function of the duration of the cardiac phase and the speed of image data acquisition

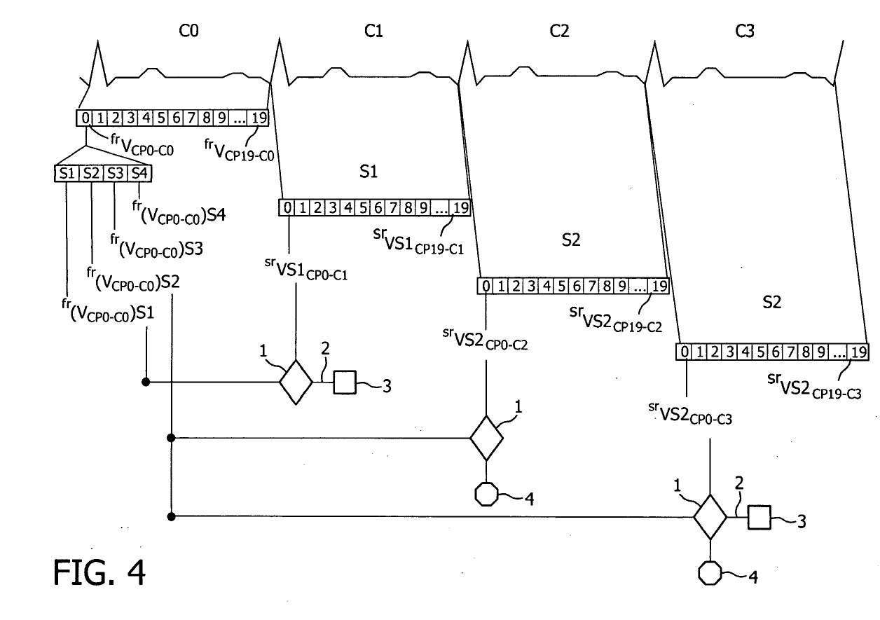

The acquisition of the image data for the number of sectors which constitute the volume is described with reference to FIG. 4. The volume (V) is defined as having sectors (VS1-VS4). Each cardiac cycle Ck is defined as having cardiac phases (CP0-CP19). Image data is acquired during for example four cardiac cycles C0-C3. Using this notation, image data ^{fr}V_{CP0-C0} for volume (V) is acquired during cardiac phase CP0 of cardiac cycle C0 with a first resolution (fr). Image data ^{fr}V_{CP1-C0}, . . . , ^{fr}V_{CP19-C0} for volume (V) are acquired during cardiac phases CP1-CP19 of cardiac cycle C0.

In the description ^{fr}(V_{CPj-C0})Si=FRsi designate the portion of data corresponding to sector (Si) and acquired for volume (V) with the first resolution during phases CP_{J} of first cardiac cycle C0. Image data ^{sr}VS1_{CP0-C1}, ^{sr}VS1_{CP1-C1}, . . . , ^{sr}VS1_{CP1-C1} for volume sector VS1 are similarly acquired with a second resolution (sr) during each of cardiac phases (CP0-CP19) of cardiac cycle C1. In the following ^{sr}VS1_{CPj-Ck}=SRi designate the image data of second resolution acquisition made on sector Si during cardiac phase (CPj) of cardiac cycle Ck.

According to the method of the invention shown on FIG. 4, the first ^{fr}(V_{CP0-C0})Si and the second ^{sr}VSi_{CP0-C0} image data of the same sector Si are compared to a determined condition to validate the three-dimensional ultrasound second image data representative of the sector when the comparison satisfy this condition.

Each SR sector (SRᵢ) is compared (1) to the FR acquisition (FRsi). In one embodiment validation (2) of the different sectors is performed by calculating the similarity Tᵢ between the image data of the FR and the SR sectors, in a sum of squared differences sense.

$${T}_{i} = {min}_{{T}_{i}} {\sum {( FRsi ( x ) - {SR}_{i} ( x ) )}^{2}}_{∀ x ∈ ( FR ⋂ SR ) }$$

If the sum does not exceed a predetermined value the image data of second resolution are saved (3) in a memory of the apparatus or directly displayed on a display. If not a new acquisition (4) of the same sector is triggered at the beginning of the following cardiac cycle, until the image data satisfy the condition or until a given number of acquisition have been unsuccessful on the same sector and in this last case a warning signal is transmitted to the operator. A warning signal may also be outputted as soon as the above-mentioned sum exceeds the predetermined value. One advantage of the method is to obtain large high-resolution image volumes without artefacts.

As an example illustrated in FIG. 4, if during the comparison between (^{fr}V_{CP0-C0})S2 and ^{sr}VS2_{CP0-C2} the above-mentioned sum exceeds the predetermined value the image data of second resolution obtained during cardiac cycle C2 are not saved (3) in a memory of the apparatus. A new acquisition is triggered (4) and realized during the following cardiac cycle C3, until the image data satisfy the condition, or a number of unsuccessful acquisitions have been attempted.

Further, if a given number of image data ^{sr}VSi_{CPj-Ck} acquired during the first phases of a cardiac cycle Ck are matching the condition and the remaining image data acquisitions during the remaining phases are not matching the condition, the system may interpolate these non-matching or invalidated acquisitions, thus avoiding to start a new acquisition for the remaining phases of the same sector during a following cardiac cycle. Actually, the image data matching the condition may be used to interpolate the image data not matching the condition. In other words, the image data not matching the condition may be re-calculated so as to be positioned at the same location than the image data matching the condition.

Alternatively, the image data not matching the condition may be re-calculated so as to position them differently until they reach the condition. In such a way, another acquisition is also avoided, the proper positioning of the sectors in the volume is realized by means of a re-calculation of the image data of each invalidated sector.

The said approach is used during a sufficient number of cardiac cycles, for instance six C0-C5. First cycle C0 is used for first resolution acquisition on the volume (V) and assuming for example that one volume sector (for example S2) needs a new acquisition, five other cycles C1-C5 are needed to acquire the four sectors (S1-S4), so that image data for the four volume sectors are acquired during each of cardiac phases CP0-CP19 of the cardiac cycles for delivering image data matching the condition.

Hence the present invention describes a robust acquisition protocol to overcome the potential junction artefacts originated in a full-volume acquisition process.

If the final difference between SR and FR image data exceeds a certain threshold, the operator may be warned that artefacts might be present in the acquisition. Moreover, a posteriori verification on the seamless transition from sector to sector can be performed by inspection of the gradient image or other similar low-level image processing algorithms.

According to an improvement of the invention, the sectors are chosen such that at least two sectors partially overlap. This situation is shown in FIG. 1B. In case of no artifact, the image data in the overlapping regions should be the same for two partially overlapping sectors. As a consequence, if the image data in an overlapping region differs from one sector to another consecutive and overlapping sector, this means that there is an artifact and that said consecutive sector should be invalidated or further processed.

According to this improvement, comparison of the overlapping portions may be performed by calculating the similarity between the sectors, in a sum of squared differences sense, as of:

$${T}_{i} = {min}_{{T}_{i}} {\sum {( {SR}_{i} ( x ) - {SR}_{i + 1} ( x ) )}^{2}}_{∀ x ∈ ( {SR}_{i} ⋂ {SR}_{i + 1} ) }$$

where T₀ is assumed to be the identity (the first sector serves as reference).

This improvement allows further improving reduction of the artefacts in the final image, as it may be used as an additional verification of the presence or absence of artefacts.

In another embodiment, the order of acquisition may also be important. If the FR acquisition is first performed, the SR sectors image data can be compared to it as long as they are acquired, without the need to wait until all acquisitions are finished. However, other acquisition orders are possible. If the FR volume is acquired in the middle, that is to say 2 SR acquisitions are performed, then the FR acquisition and then the remaining 2 SR acquisitions, the LR acquisition will be closer to all SR acquisitions, thus reducing the probability of artefacts in case there is a continuous drift in the acquisitions.

By obtaining a three-dimensional image representing the heart in each of the cardiac phases, a variety of information can be obtained. The three-dimensional images of the heart at successive cardiac phases can be displayed as a function of time to represent heart movement. The moving image can be used to identify end systole and end diastole and to perform other diagnostics. Images for a selected cardiac phase can be rotated to a desired orientation for improved analysis. Image analysis techniques can be utilized to quantify maximum and minimum volumes of the left ventricle. From this information, ejection volume and ejection fraction can be calculated.

The invention is not limited to a calculation of the similarity based on the sum of squared differences. Other similarity metrics could be used, such as the sum of the absolute differences, normalized correlation or normalized mutual information.

Any reference sign in the following claims should not be construed as limiting the claim. It will be obvious that the use of the verb “to comprise” and its conjugations does not exclude the presence of any other elements besides those defined in any claim. The word “a” or “an” preceding an element does not exclude the presence of a plurality of such elements.

1. A method for medical ultrasound imaging, comprising the steps of:
a) acquiring with a first resolution a first ultrasound image data representative of a volume of an interest organ during a cardiac cycle of a patient;
b) acquiring with a second resolution higher than the first resolution a second ultrasound image data representative of a three-dimensional sector of said volume during another cardiac cycle of the patient;
c) comparing the first and the second ultrasound image data of said sector so as to detect a similarity between the first and the second ultrasound image data and, as a function of said comparison,
validating the three-dimensional ultrasound second image data if the first and the second ultrasound image data are substantially similar or otherwise
further processing said sector.

2. A method for medical ultrasound imaging as claimed in claim 1 wherein the step of comparing the first and the second ultrasound image data comprises calculating their similarity according to one of a sum of squared differences, sum of the absolute differences, normalized correlation or normalized mutual information.

3. A method for medical ultrasound imaging as claimed in claim 1 wherein the step of validating the data comprises registering in a memory or displaying on a display the second ultrasound image data of the sector.

4. A method for medical ultrasound imaging as claimed in claim 1, wherein the step of further processing the sector comprises either acquiring a new second resolution ultrasound image data of said sector and iterating the comparing step or interpolating the three dimensional ultrasound second image data.

5. A method for medical ultrasound imaging as claimed in claim 1 wherein the volume is a whole patient's cardio-vascular organ and the method comprises acquiring with the second resolution a plurality of second ultrasound image data representative of a plurality of sectors during a plurality of cardiac cycles of the patient, said plurality of sectors covering the whole patient's cardio-vascular organ.

6. A method for medical ultrasound imaging as claimed in claim 5 wherein at least two sectors partially overlap.

7. A method for medical ultrasound imaging as claimed in claim 5 wherein the step of acquiring a volume or a sector comprises the acquisition of image data during several phases of a cardiac cycle and the step of comparing compares the image data of a sector acquired with a first resolution during a cardiac phase to the image data of the same sector acquired with a second resolution during the same cardiac phase.

8. A method for medical ultrasound imaging as claimed in claim 1 wherein the step of further processing comprises a step of outputting a warning signal.

9. Apparatus for medical ultrasound imaging, comprising:
an acquisition system arranged for:
acquiring with a first resolution a first ultrasound image data representative of a volume of an interest organ during a cardiac cycle of a patient;
acquiring with a second resolution higher than the first resolution a second ultrasound image data representative of a three-dimensional sector of said volume during another cardiac cycle of the patient; and
a comparator for comparing the first and the second ultrasound image data of said sector.

10. A computer program for controlling a medical ultrasound imaging apparatus, said computer program implementing:
means for controlling the acquisition with a first resolution of a first ultrasound image data representative of a volume of an interest organ during a cardiac cycle of a patient;
means for controlling the acquisition with a second resolution higher than the first resolution of a second ultrasound image data representative of a three-dimensional sector of said volume during another cardiac cycle of the patient; and
means for comparing the first and the second ultrasound image data of said sector.
